(19) United States
(12) Patent Application Publication
(10) Pub. No.: US 2026/0283733 A1
(43) Pub. Date: Sep. 24, 2026

(54) DEVICE FOR POSITIONING A SCREEN BASKET IN A CONTAINER

(71) Applicant: Aesculap AG, Tuttlingen (DE)

(72) Inventors: Arne Jansen-Troy, Stockach (DE); Allan Maas, Konstanz (DE); Daniel Kiessling, Villingen-Schwenningen (DE); Johannes Hermann, Schoemberg (DE)

(21) Appl. No.: 19/475,496

(22) PCT Filed: Apr. 17, 2024
(86) PCT No.: PCT/EP2024/060339

§ 371 Date: Oct. 15, 2025

(30) Foreign Application Priority Data
Apr. 18, 2023 (DE) 10 2023 109 767.9

(51) Int. Cl. A61B 50/34; A61B 90/70; A61B 90/96; A61B 90/98

(52) U.S. Cl. CPC A61B 50/34; A61B 90/70; A61B 90/96; A61B 90/98

(57) ABSTRACT

A device for positioning a screen basket in a container has a mechanical interface for limiting movement of the screen basket in the container. The device can also have a mechanical interface for gripping the device. The device can further have a machine-readable interface for detecting the screen basket and/or a machine-readable interface for detecting the position and/or orientation of the screen basket, for example in a coordinate system for navigation or positioning purposes. A system can feature the device and a container with a trough for receiving the screen basket. The device can be inserted into the trough of the container. The mechanical interface for limiting movement of the screen basket in the container rests against the inner wall of the trough and braces the screen basket against the trough, thus fixing the position of the screen basket in the container.

### CROSS-REFERENCE TO RELATED APPLICATIONS

This application is the United States national phase entry of International Application No. PCT/EP2024/060339, filed on Apr. 17, 2024, and claims priority to German Application No. 10 2023 109 767.9, filed on Apr. 18, 2023.

### FIELD

The invention relates to a device for positioning a screen basket in a container.

### BACKGROUND

DE 10 2020 131104 A1 discloses a sterilizing screen tray with a fixing handle, which is designed and configured for insertion into and removal from a container trough.

### SUMMARY

A device for positioning a screen basket in a container has a mechanical interface to the container, which is configured for limiting the movement of the screen basket relative to the container. The device has:
a machine-readable interface, visible in a top view, for detecting the screen basket and/or
a machine-readable interface, visible in a top view, for detecting the position and/or orientation of the screen basket, in particular in a coordinate system for navigation or positioning purposes.

The machine-readable interfaces, visible in a top view, are in particular visible from outside the container.

The machine-readable interface for detecting the screen basket and/or its position and/or its orientation allows for improved gripping and positioning by a robot.

In one embodiment, the device has a mechanical interface for gripping the device.

In one embodiment, the device has a mechanical interface for connection to the screen basket, in particular a lug and/or a locking mechanism for force-fitting and/or form-fitting connection to the screen basket, and/or a joint for adhesive bonding and/or screwing to the screen basket. This device represents a screen basket positioning element that can be arranged on a conventional screen basket without such a screen basket positioning element. The screen baskets themselves do not require adjustment.

In one embodiment, the device has at least one flushing channel leading through the device, in particular in the region of the lug and/or the locking mechanism. This makes the device particularly suitable for use in a sterilization container.

In one embodiment, the screen basket and the device are formed in one piece. This means that the screen basket itself includes the function of the screen basket positioning element.

Preferably, the gripping interface comprises the machine-readable interface for detecting the screen basket and/or the machine-readable interface for detecting the position and/or orientation of the screen basket. This arrangement is particularly compact.

Preferably, the mechanical interface to the container comprises a surface for reducing play between the screen basket and the container. This reduces wear on the screen basket and the container.

Preferably, the device has a surface which comprises the machine-readable interface for detecting the screen basket and/or the machine-readable interface for detecting the position of the screen basket.

In one embodiment, the machine-readable interface for detecting the screen basket and/or the machine-readable interface for detecting the position of the screen basket and/or orientation is embossed into the surface or printed on the surface. This device is particularly easy to mass produce.

In one embodiment, the surface is configured for stacking a further screen basket. This allows a plurality of screen baskets to be arranged one above the other.

In one embodiment, the mechanical interface for gripping comprises the surface. This allows for better gripping with the robot.

Preferably, the machine-readable interface for detecting the screen basket comprises an optically readable marker, in particular a barcode, a data matrix code, alphanumeric data or a QR code, which encodes an identifier of the screen basket in a machine-readable manner.

In one embodiment, the machine-readable interface for detecting the position and/or orientation comprises a symbol with a unique indication of an orientation of the symbol. This allows for an unambiguous indication of orientation.

It can be provided that the device is made at least partially of plastics or at least partially of metal.

It can be provided that the device comprises a transmitter which is configured to transmit an electromagnetic signal, in particular a Bluetooth Low Energy signal or a Radio Frequency Identification signal, for detecting the screen basket, wherein the machine-readable interface for detecting the screen basket comprises the transmitter and/or that the device comprises a transmitter which is configured to transmit an electromagnetic signal, in particular a Bluetooth Low Energy signal or a Radio Frequency Identification signal, for detecting the position and/or orientation of the screen basket, wherein the machine-readable interface for detecting the position and/or orientation comprises the transmitter.

It can be provided that the transmitter is adhesively bonded to the device or that the device is at least partially made of plastics, wherein the transmitter is surrounded by the plastics.

The invention further relates to a system, wherein the system comprises the device and a container, wherein the container comprises a trough for receiving the screen basket, wherein the device can be inserted into the trough of the container and is adapted such that the mechanical interface for limiting the movement of the screen basket in the container rests against an inner wall of the trough and braces the screen basket against the trough, thus fixing the position of the screen basket in the container.

In this respect, the invention relates in particular to a device serving as a screen basket positioning element with which a screen basket can be retrofitted, to such a device which is already attached to the screen basket in the delivery state and to such a device which is formed with the screen basket.

### BRIEF DESCRIPTION OF THE DRAWINGS

Further advantageous embodiments can be found in the following description and drawings, in which:

FIG. 1A is a perspective view of a screen basket positioning element;

FIG. 1B is a view of the underside of the screen basket positioning element;

FIG. 1C shows a detail view of Section A in FIG. 1B;

FIG. 2A is a top view of a screen basket with two screen basket positioning elements in a sterile goods container;

FIG. 2B shows a detail of the top view;

FIG. 3 is a perspective view of two stacked screen baskets, each with two screen basket positioning elements; and

FIG. 4 is a perspective view of a screen basket with two screen basket positioning elements inserted into a sterile goods container.

### DETAILED DESCRIPTION

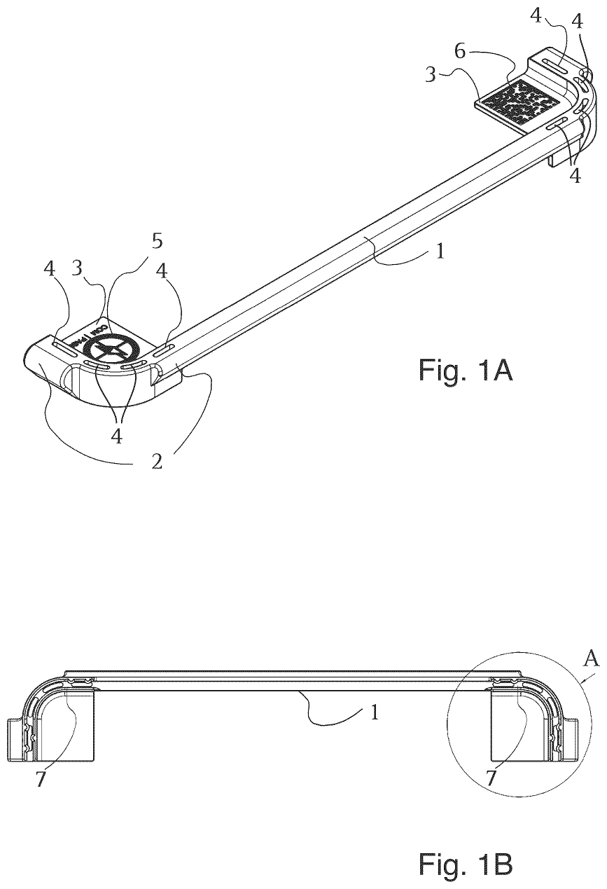

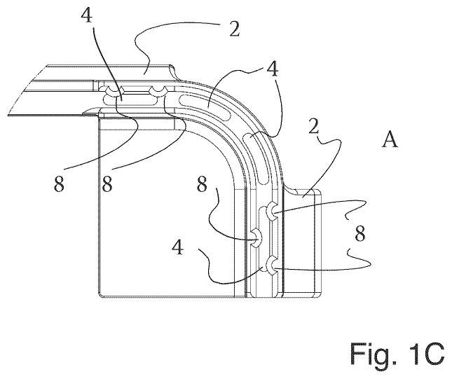

FIG. 1A shows a screen basket positioning element 1. The screen basket positioning element 1 is an example of a device for positioning a screen basket in a container. In the example, the container is a sterile goods container.

The screen basket positioning element 1 can be arranged on the screen basket. In the example, the screen basket positioning element 1 can be arranged on an upper edge of the screen basket.

It may be provided that the screen basket positioning element is instead formed in one piece with the screen basket.

The screen basket positioning element 1 can be made of plastics or metal. It can be provided that the screen basket positioning element 1 is made partially of plastics and/or partially of metal.

The screen basket positioning element 1 comprises a first mechanical interface 2, which is configured for limiting the movement of the screen basket in the sterile goods container. The first mechanical interface 2 is an interface to the sterile goods container. In the example, the first mechanical interface 2 comprises surfaces for reducing play between the screen basket and the sterile goods container. In the example, the surfaces are arranged on at least one outer edge portion of the screen basket positioning element 1. In the example, three surfaces are provided. It can be provided that the first mechanical interface 2 comprises one, two or more than three surfaces.

The screen basket positioning element 1 comprises a second mechanical interface 3 which is configured to grip the screen basket positioning element 1. The second mechanical interface 3 is a gripping interface. In the example, the second mechanical interface 3 comprises two surfaces. It can be provided that the second mechanical interface 3 comprises one or more than two surfaces.

In the example, the screen basket positioning element 1 has two corner pieces and a web that connects the two corner pieces. In the example, the corner pieces are configured as lateral stops for a gripper for gripping the screen basket positioning element 1. In the example, the corner pieces each encompass one of the two gripping surfaces.

In one embodiment, the screen basket positioning element 1 has at least one flushing channel 4. In the example, four spaced-apart flushing channels are provided for each corner piece. In the example, the flushing channels are configured as elongated holes. The flushing channels can also have a different shape. More or fewer than four flushing channels may be provided.

In one embodiment, the screen basket positioning element 1 comprises a first machine-readable interface 5 for detecting a position and/or orientation of the screen basket. The first interface 5 indicates the position and/or orientation of the screen basket. A coordinate system for navigation or positioning purposes is, for example, fixed in relation to the sterile goods container. Based on the detected position of the first machine-readable interface 5, the position of the screen basket is known. Based on the detected orientation of the first machine-readable interface 5, the orientation of the screen basket is known.

In the example, the first machine-readable interface 5 is printed on one of the surfaces for the gripper. It can also be provided that the first machine-readable interface 5 is embossed into this surface.

It can be provided that the side of the surface facing away from the first machine-readable interface 5 comprises one or more undercuts. This allows for secure gripping or holding of the screen basket when acceleration occurs due to a change in the direction of movement.

In one embodiment, the first machine-readable interface 5 comprises a symbol with a unique indication of an orientation of the symbol. In the example, the symbol comprises a circle with diagonals crossing at right angles, each parallel to two of the side edges of the screen basket, and within which a compass needle is shown at a defined angle to the diagonals. A different symbol may also be provided. For example, the symbol includes an arrow that runs parallel to one of the side edges or at a defined angle to the side edges. Based on the detected orientation of the second machine-readable interface 5, the orientation of the screen basket is known.

By determining the position, the position and/or orientation of the screen basket relative to the sterile goods container can be determined. It may be provided that the height of the screen basket relative to the sterile goods container is not fixed.

In one embodiment, the first machine-readable interface 5 comprises a first transmitter. The first transmitter is configured to transmit a first electromagnetic signal for detecting a position and/or orientation of the screen basket. The first electromagnetic signal is, for example, a Bluetooth Low Energy signal or a Radio Frequency Identification signal.

It can be provided that the first machine-readable interface 5 comprises the symbol and the first transmitter.

In one embodiment, the screen basket positioning element 1 comprises a second machine-readable interface 6 for detecting the screen basket.

In the example, the second machine-readable interface 6 is printed on the other of the surfaces for the gripper. It can also be provided that the second machine-readable interface 6 is embossed into this surface.

It can be provided that the side of the surface facing away from the second machine-readable interface 6 comprises one or more undercuts. This allows for secure gripping or holding of the screen basket when acceleration occurs due to a change in the direction of movement.

In one embodiment, the second machine-readable interface 6 comprises an optically readable marker which encodes an identifier of the screen basket in a machine-readable manner. The optically readable marker is, for example, a barcode or a data matrix code or a QR code. In one embodiment, the optically readable marker comprises alphanumeric data.

In one embodiment, the second machine-readable interface 6 comprises a second transmitter. The second transmitter is configured to transmit a second electromagnetic signal for detecting the screen basket. The second electromagnetic signal is, for example, a Bluetooth Low Energy signal or a Radio Frequency Identification signal.

It can be provided that the first machine-readable interface 5 and the second machine-readable interface 6 are arranged on a common surface.

It may be provided that instead of the first transmitter and the second transmitter, a common transmitter is provided which transmits the first and the second electromagnetic signal.

The common transmitter or the first transmitter or the second transmitter can be adhesively bonded to the device 1. It can be provided that the device 1 is at least partially made of plastics, and the common transmitter or the first transmitter or the second transmitter is surrounded by the plastics.

FIG. 1B is a view of an underside of the screen basket positioning element 1. The screen basket positioning element 1 has a third mechanical interface 7, which is configured to connect the screen basket positioning element 1 to the screen basket. The third mechanical interface 7 is an interface to the screen basket. The third mechanical interface 7 is omitted if the screen basket and the screen basket positioning element 1 are in one piece.

In this case, where the screen basket positioning element 1 and screen basket 9 are in one piece, the two machine-readable interfaces 5 and 6 are provided on the upper side of tabs. The upper side is oriented opposite to the underside shown in FIG. 1B. In the case of such a one-piece configuration, the tabs can be made from the same sheet metal as the screen basket 9 or a screen basket wall. For production purposes, these tabs can, in particular, be cut from the same sheet metal as the screen basket 9 or the screen basket wall. After cutting out the tabs, they are then bent inwards. The “inwards” direction means that the tabs are aligned at least substantially in one plane, facing one another. The term “facing one another” can also be interpreted as “in the direction of an orifice of the screen basket.” This plane lies at least approximately in the region of the orifice of the screen basket 9.

FIG. 1C shows a detail A of the view. Detail A includes one of the two corner pieces. In the example, the two corner pieces are of identical design.

In the example, the third mechanical interface 7 comprises six lugs 8 per corner piece for force-locking and/or form-locking connection to the screen basket. More or fewer than four lugs may be provided. The corner piece, for example, has two legs with a groove. On one leg, two lugs are arranged at the edge of the groove, projecting into the groove. On one leg, two lugs are arranged at the edge of the groove, projecting into the groove, with one lug projecting into the groove on the opposite side of the groove between the two lugs. The groove is configured to receive the upper edge of the screen basket.

In the example, a flushing channel 4 leading through the device 1 is arranged in the region of each lug.

The third mechanical interface 7 may additionally or instead have a locking mechanism for force-locking and/or form-locking connection to the screen basket.

The third mechanical interface 7 can additionally or instead have a joint for adhesive bonding.

The third mechanical interface 7 can additionally or instead have a joint for screwing to the screen basket.

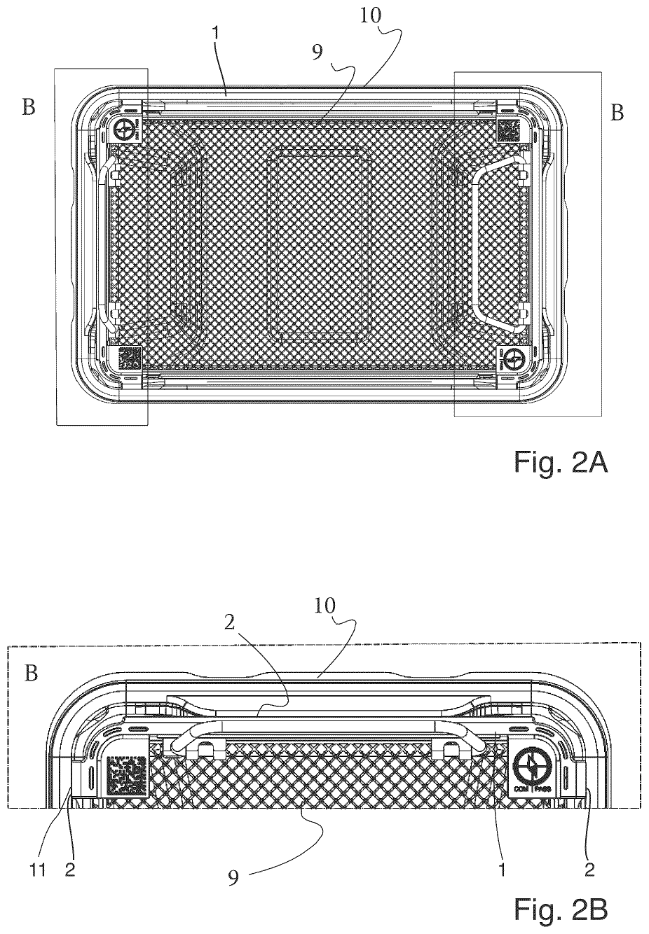

FIG. 2A is a top view of an exemplary screen basket 9 with two screen basket positioning elements 1 in a sterile goods container 10. In the example, two identical screen basket positioning elements 1 are provided. In the example, the screen basket positioning elements 1 are arranged on opposite sides of the sterile goods container 10.

In the example, one screen basket positioning element 1 is arranged on each short side of the screen basket 9. The corner pieces are each arranged at a corner of the screen basket 9.

The first machine-readable interface 5 and the second machine-readable interface 6 are arranged so that they are visible in the top view of the sterile goods container 10 from outside the sterile goods container 10.

FIG. 2B shows a detail B of the top view, which includes the screen basket positioning element 1.

In the example, the first mechanical interface 2 touches an inner wall 11 of a trough of the sterile goods container 10. In the example, the surfaces for reducing play between the screen basket 9 lie on the inner wall 11 of the sterile goods container 10.

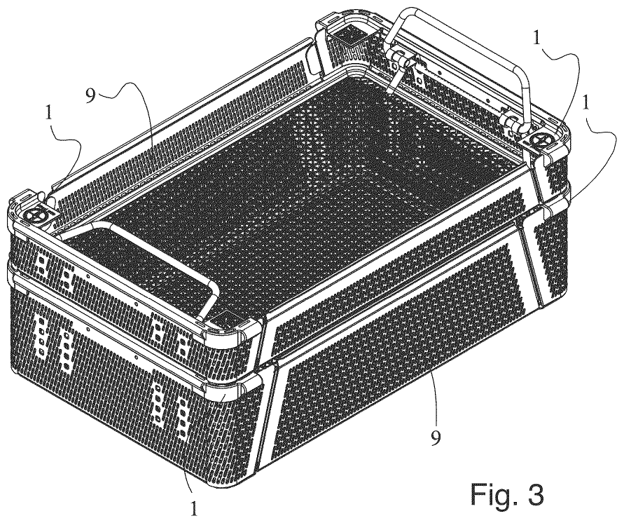

FIG. 3 is a perspective view of two stacked screen baskets 9, each with two screen basket positioning elements 1. In the example, the screen baskets 9 are of different heights. In the example, the upper screen basket 9 stands on the stacking surface provided on the lower screen basket 9.

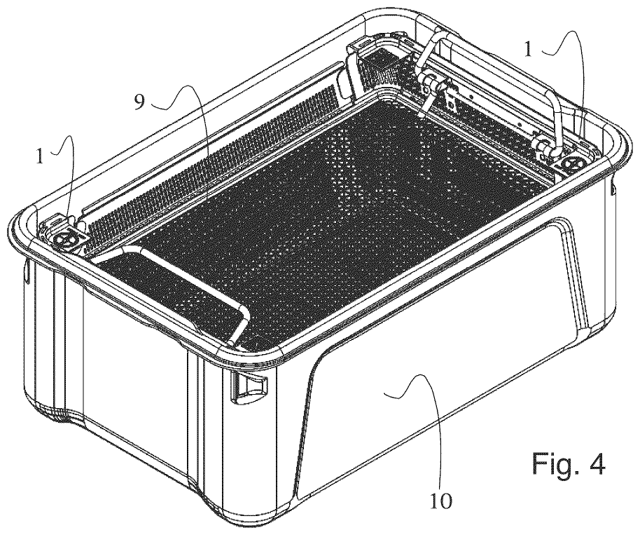

FIG. 4 is a perspective view of the screen basket 9 with two screen basket positioning elements 1, which screen basket is inserted into the sterile goods container 10.

In this system, the screen basket positioning element 1 can be inserted into the trough of the container 10. The screen basket positioning element 1 is adapted such that the first mechanical interface 2 for limiting the movement of the screen basket 9 in the container rests against an inner wall 11 of the trough and braces the screen basket 9 against the trough, thus fixing the position of the screen basket in the container 10.

1.-18. (canceled)

19. A device for positioning a screen basket in a container, the device comprising a first mechanical interface to the container, the first mechanical interface being configured for limiting movement of the screen basket relative to the container, the device further comprising at least one of:
a first machine-readable interface visible in top view for detecting the screen basket, or
a second machine-readable interface visible in top view for detecting a position and/or orientation of the screen basket.

20. The device according to claim 19, further comprising a second mechanical interface for gripping the device.

21. The device according to claim 20, wherein the second mechanical interface for gripping the device comprises the first machine-readable interface and/or the second machine-readable interface.

22. The device according to claim 19, further comprising a third mechanical interface for connection to the screen basket.

23. The device according to claim 22, wherein the third mechanical interface comprises a lug and/or a locking mechanism for force-fitting and/or form-fitting connection to the screen basket.

24. The device according to claim 22, wherein the third mechanical interface comprises a joint for adhesive bonding and/or screwing to the screen basket.

25. The device according to claim 19, further comprising at least one flushing channel leading through the device.

26. The device according to claim 19, wherein the device comprises the screen basket, and the device and the screen basket are formed in one piece.

27. The device according to claim 19, wherein the device comprises both the first machine-readable interface and the second machine-readable interface, the first machine-readable interface and the second machine-readable interface provided on tabs that are cut from sheet metal in one piece with the screen basket and bent inwards.

28. The device according to claim 19, wherein the first mechanical interface comprises a surface for reducing play between the screen basket and the container.

29. The device according to claim 19, further comprising an interface surface that contains the first machine-readable interface and/or the second machine-readable interface.

30. The device according to claim 29, wherein the first machine-readable interface and/or the second machine-readable interface is embossed into the interface surface or printed on the interface surface.

31. The device according to claim 29, wherein the interface surface is configured for stacking a further screen basket.

32. The device according to claim 29, wherein the interface surface comprises a second mechanical interface for gripping the device.

33. The device according to claim 19, wherein the first machine-readable interface comprises an optically readable marker that encodes an identifier of the screen basket in a machine-readable manner.

34. The device according to claim 19, wherein the second machine-readable interface comprises a symbol with a unique indication of an orientation of the symbol.

35. The device according to claim 19, further comprising a transmitter configured to transmit an electromagnetic signal for detecting the screen basket, wherein the first machine-readable interface comprises the transmitter.

36. The device according to claim 19, further comprising a transmitter configured to transmit an electromagnetic signal for detecting the position and/or orientation of the screen basket, wherein the second machine-readable interface comprises the transmitter.

37. The device according to claim 19, further comprising a transmitter configured to transmit an electromagnetic signal for detecting at least one of the screen basket, the position of the screen basket, or the orientation of the screen basket, wherein the transmitter is adhesively bonded to the device or wherein the device is at least partially made of plastics, with the transmitter surrounded by the plastics.

38. A system comprising:
a container comprising a trough for receiving a screen basket; and
a device for positioning the screen basket in the container;
the device being insertable into the trough of the container and adapted such that a mechanical interface for limiting movement of the screen basket in the container rests against an inner wall of the trough and braces the screen basket against the trough, thus fixing a position of the screen basket in the container,
the device comprising at least one of:
a first machine-readable interface visible in top view for detecting the screen basket, or
a second machine-readable interface visible in top view for detecting the position and/or an orientation of the screen basket.
